(19) United States
(12) Patent Application Publication
(10) Pub. No.: US 2011/0098351 A1
(43) Pub. Date: Apr. 28, 2011

(54) Lactone compounds which can be used as antioxidant agents in pharmaceutical, cosmetic or food compositions and their method of preparation

(75) Inventors: Charles Mioskoski, Strasbourg (FR); Thierry Le Gall, Chatillon (FR); Marine Desage, Paris (FR); Stephane Meunier, San Diego, CA (US); Frederic Taran, Gif-Sur-Yvette (FR); Stephanie Nowaczyk, Chaville (FR)

(73) Assignees: COMMISSISSARIAT A L'ENERGIE ATOMIQUE; CENTRE NATIONAL de la RECHERCHE SCIENTIFIQUE

(21) Appl. No.: 10/519,764

(22) PCT Filed: Jun. 28, 2003
(86) PCT No.: PCT/FR03/01979

§ 371 Date: Jan. 18, 2006

(30) Foreign Application Priority Data
Jun. 28, 2002 (FR) 02/08081

(51) Int. Cl. A61K 31/343; C07D 307/60; C07D 307/83; A61K 31/341; A61P 39/06

(52) U.S. Cl. 514/468; 549/477; 549/458; 514/473

(57) ABSTRACT

The subject of the present invention is specific lactone compounds which can be used as antioxidant agents in pharmaceutical or cosmetic compositions or in food products, the said compounds containing a naphthalene ring onto which two lactone units are grafte.

### TECHNICAL FIELD

The present invention relates to specific lactone compounds which can be used as antioxidant agents for the manufacture of antioxidant compositions, in particular pharmaceutical, cosmetic or food compositions.

The present invention also relates to a method for preparing such compounds.

The general field of the invention is therefore that of antioxidants.

### STATE OF THE ART

Antioxidants have the characteristic feature of capturing free radicals, which are very reactive molecules involved in numerous pathologies, in particular pathologies resulting from oxidant stress, such as for example inflammatory diseases, cardiovascular diseases or diabetes.

Accordingly, certain antioxidants may be used for their anti-inflammatory activity, in particular antioxidants which make it possible to inhibit the production of proinflammatory cytokines such as the factor TNF-α, in the macrophages and the monocytes.

Antioxidants may also be involved in protecting cells, by limiting the onset of the genetic programme of cell death or apoptosis, which can be caused by an accumulation of free radicals.

Several antioxidants of natural origin have also been the subject of evaluations regarding their action against cancer. Accordingly, in the article by M. Jang et al., “Cancer Chemoprotective Activity of Resveratrol, a natural product derived from grapes”, Science 1997, 275, 218-220 [1], resveratrol, an antioxidant phytoalexin extracted from grapes is described as exhibiting a preventive activity against cancer on animal models. The article by M. V. Eberhardt et al. “Antioxydant Activity of Fresh Apples”, Nature 2000, 405, 903-904 [2] demonstrates that apple extracts tested on anticancer cell lines likewise cause an antiproliferative activity in vitro on these lines.

Finally, studies such as those published in the article by T. Finkel et al., “Oxydants, Oxidative Stress and the biology of ageing”, Nature 2000, 408, 239-247 [3], have also demonstrated the relationships between oxidant stress and cellular ageing. As a result, it is possible to envisage incorporating antioxidants into cosmetic compositions, intended to capture the free radicals responsible in particular for the appearance of wrinkles.

### DISCLOSURE OF THE INVENTION

The aim of the present invention is to provide specific lactone compounds, some of which are novel, which can be used in a particularly efficient manner as antioxidant agents for the manufacture of antioxidant compositions such as pharmaceutical, cosmetic and food compositions.

The aim of the present invention is to also provide a method for preparing lactone compounds in accordance with the invention.

Accordingly, the present invention relates, according to a first object, to compounds corresponding to the following formula (I):

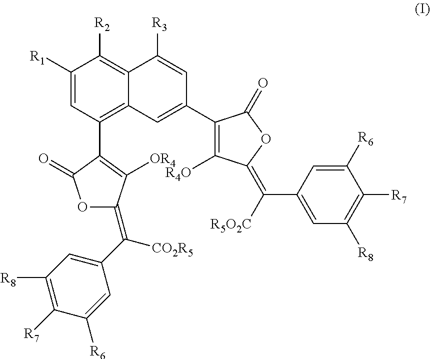

in which:
R₁, R₆, R₇ and R₈, which are identical or different, represent H, —OH or —OR₉;
R₂ represents H, —OH or —OR₉; R₃ represents H, R₉, —CO₂R₉ or —CO—NHR₁₀; or R₂ and R₃ form together —O—CO—;
R₄ and R₅, which are identical or different, represent H or R₉;
R₉ represents a linear or branched alkyl group containing from 1 to 20 carbon atoms;
R₁₀ represents R₉ or a group —(CH₂)ₐ—NH—(CH₂)_{b}—NH₂, with a and b, which are identical or different, being integers ranging from 2 to 4;
   and the salts of these compounds;
   with the exception:
of the compound for which R₂ and R₃ form together a group —OCO—, R₄, R₅, R₆ and R₈ represent H, R₁ and R₇ represent —OH and the disalts of potassium corresponding to this compound;
of the compound in which R₂ and R₃ form together a group —O—CO—, R₁ and R₇ represent —OCH₃, R₄ and R₅ represent —CH₃ and R₆ and R₈ represent H;
of the compound in which R₁, R₂ and R₇ represent —O—CH₃, R₃ represents —CO₂CH₃, R₄ and R₅ represent CH₃ and R₆ and R₈ represent H.

As is mentioned above, the invention also comprises the possible salts corresponding to the compounds as defined above.

The expression salts is understood to mean, in the preceding text and in the text which follows, the ionic compounds resulting from the action of an inorganic base on the labile proton(s) of a compound of formula (I).

Accordingly, when R₅ represents H in the compound according to the invention, the corresponding salt is in the following form:

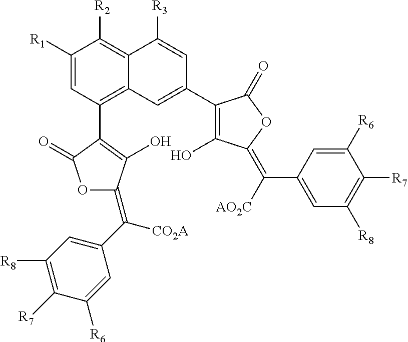

When R₄ represents H in the compound according to the invention, the corresponding salt is in the following form:

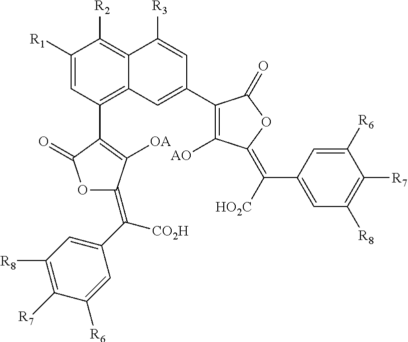

it being possible for the symbol A to represent, for the two salt forms mentioned above, an alkali metal such as Na⁺, K⁺ or an ammonium cation NH₄⁺.

It is specified that, for the first excluded compound, the disalts of potassium are in particular those represented by formula (XII) below.

It is specified that, according to the invention, in the preceding text and in the text which follows, the expression linear or branched alkyl group containing from 1 to 20 carbon atoms is understood to mean an unsaturated hydrocarbon group such as a methyl, ethyl, propyl, isopropyl, butyl, isobutyl or tert-butyl group.

In formula (I), when R₂ and R₃ form together a group —O—CO—, the compound corresponds to the following formula (II):

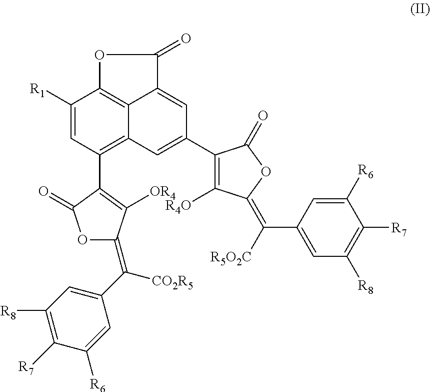

R₁, R₄, R₅, R₆, R₇ and R₈ corresponding to the same definition as that given above. These compounds, in particular by virtue of the presence of a central cyclic unit with a lactone ring, are characterized by a particularly effective antioxidant power.

In formula (I), R₂ and R₃ may also independently form identical or different radicals such as hydrogen atoms. By way of example, there may be mentioned the following compound of formula (III):

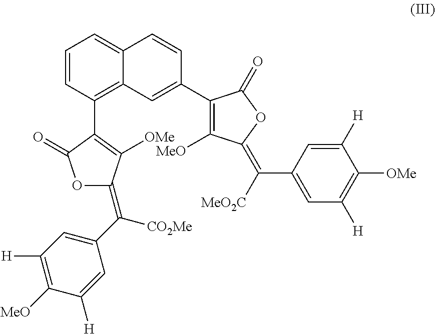

in which R₁, R₂, R₃, R₆ and R₈ represent H, R₄ and R₅ represent CH₃ (designated by Me in the formula above), R₇'s represent —OCH₃ (designated by —OMe in the formula above).

As regards the group R₉, which represents a linear or branched alkyl group containing from 1 to 20 carbon atoms, there may be mentioned, by way of example, the methyl, ethyl, n-propyl, n-butyl or t-butyl group.

The subject of the present invention is also a method for preparing compounds of the following formula (I):

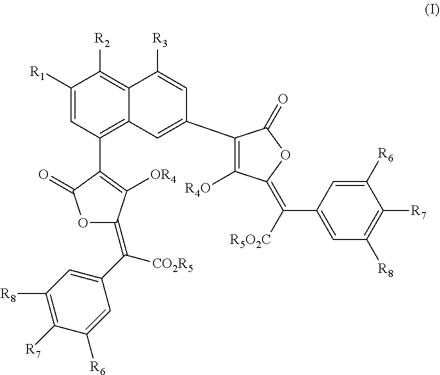

in which:
R₁, R₆, R₇ and R₈, which are identical or different, represent H, —OH or —OR₉;
R₂ represents H, —OH or —OR₉; R₃ represents H, R₉, —CO₂R₉ or —CO—NHR₁₀; or R₂ and R₃ form together —O—CO—;
R₄ and R₅, which are identical or different, represent H or R₉;
R₉ represents a linear or branched alkyl group containing from 1 to 20 carbon atoms;
R₁₀ represents R₉ or a group —(CH₂)ₐ—NH—(CH₂)_{b}—NH₂, with a and b, which are identical or different, being integers ranging from 2 to 4;
   and the salts of these compounds,
   the said method comprising successively:
a step consisting in reacting a compound of the following formula (IV):

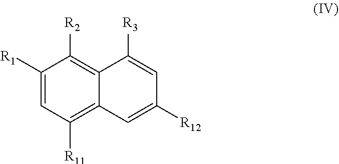

in which:
R₁, R₂ and R₃ have the same definition as that given above;
R₁₁ and R₁₂ independently represent —B(OR₁₃)(OR₁₄) or —Sn(R₁₅)₃;
R₁₃ and R₁₄, which are identical or different, represent H or an alkyl group of 1 to 7 carbon atoms or R₁₃ and R₁₄ form together a linear or branched alkylene group;
R₁₅ represents a methyl or butyl group,
   with a compound of the following formula (V):

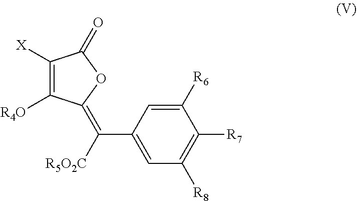

in which:
R₄, R₅, R₆, R₇ and R₈ correspond to the same definition as that given above;
X represents a leaving group,
   the said reaction being carried out in the presence of a base and a catalyst based on platinum or palladium; and
optionally a step of treatment intended to obtain a salt corresponding to the compound of formula (I). When R₁₃ and R₁₄ form together a linear or branched alkylene group, this group comprises for example from 2 to 3 carbon atoms, such as the groups —CH₂—CH₂—, —CH₂—CH₂—CH₂—, —C(CH₃)₂—C(CH₃)₂—, —CH(Ph)—CH₂—CH(Ph)—, Ph representing a phenyl group.

Preferably, the leaving group X is chosen from halogens such as F, Cl, Br, I, the triflate —O—SO₂CF₃, the group of formula —O—SO₂—(CF₂)ₙ—CF₃ with n being an integer ranging from 1 to 8.

According to the invention, the catalyst based on platinum or palladium is chosen so as to obtain a coupling reaction between the compound of formula (IV) and the compound of formula (V). Preferably, this catalyst is a platinum or palladium complex, such as dichlorobis(triphenylphosphine)palladium PdCl₂(PPh₃)₂ or tetrakis(triphenylphosphine)palladium Pd(PPh₃)₄.

According to the invention, the base used in the context of this method is a base chosen for example from NaOH, Ba(OH)₂, Na₂CO₃, K₂CO₃, Cs₂CO₃, K₃PO₄, CH₃COONa, CH₃COOK, CH₃ONa, CH₃CH₂ONa, and amines such as triethylamine.

The optional step intended for obtaining the corresponding salts consists, once the compound of formula (I) has been obtained, in treating this product, for example by causing an inorganic base to react with it. Accordingly, when R₅'s represent H, a treatment with a potassium hydroxide solution makes it possible to obtain the corresponding disalt of potassium which is none other than a potassium dicarboxylate.

It should be noted that the method according to the invention may optionally comprise steps for protecting the functional groups which are sensitive to the reaction conditions, these functional groups then being deprotected at the end of the said method. These protection and deprotection steps are steps within the capability of persons skilled in the art.

In a more detailed manner, the method of preparing the compounds of formula (I) may be carried out in the following manner.

In a first stage, the reagents corresponding to formulae (IV) and (V) are mixed with a suitable catalyst as mentioned above, in an aprotic solvent, for example tetrahydrofuran (THF), under an inert gas atmosphere.

Next, after homogenizing the reaction mixture, a base, for example sodium bicarbonate, is introduced.

The reaction mixture is then heated under reflux, with vigorous stirring, for a suitable period (that is to say the period necessary for the production of the compound of formula (I), it being possible to monitor the progress of the reaction by conventional techniques such as thin-layer chromatography).

The reaction mixture is then treated by adding water. The aqueous phase is extracted with an organic solvent, for example dichloromethane. The organic phases are combined, dried and then concentrated. The product obtained is finally purified by conventional techniques such as column chromatography.

The method for preparing the compounds in accordance with the invention uses compounds of formula (IV) and (V), which may be commercially available or which may be prepared before carrying out the method of the invention.

Accordingly, the compound of formula (IV), with R₁₁ and R₁₂ representing —B(OR₁₃)(OR₁₄), may be obtained by reacting a naphthalene-derived compound of formula (VI):

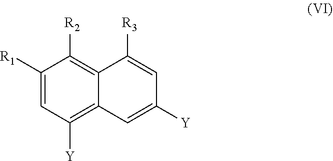

in which:
R₁, R₂ and R₃ have the same definition as that given above;
the Y's, which are identical or different, represent leaving groups chosen for example from halogens such as fluorine, chlorine, bromine, iodine, the triflate —O—SO₂—CF₃,
   with a boron compound corresponding to one of the following formulae:

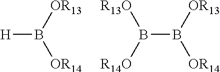

R₁₃ and R₁₄ having the same meaning as that given above,

the said reaction being carried out in the presence of a base and a catalyst based on platinum or palladium such as [1,1′-bis(diphenylphosphino)ferrocene]dichloro-palladium or PdCl₂(dppf), dppf meaning 1,1′-bis(diphenylphosphino)ferrocene.

The compounds of formula (V), for its part, may be obtained by a method comprising the following succession of steps:
a) reaction of a phenyl acetate of the following formula (VII):

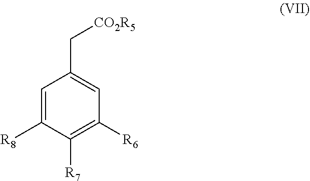

R₅, R₆, R₇ and R₈ having the same definition as that given above, in a basic medium, with an alkyl α-alkoxyacetate of formula R₄O—CH₂—CO-OAlk, R₄ corresponding to the same definition as that given above, the Alk group being a linear or branched alkyl group containing from 1 to 20 carbon atoms, at the end of which a compound of the following formula (VIII) is obtained:

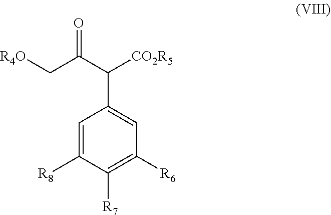

b) reaction of the compound (VIII), in a basic medium, with a silylated compound of formula (R₁₆)₃SiHal, R₁₆ being a linear or branched alkyl group containing from 1 to 4 carbon atoms, Hal being a halogen group such as F, Cl, Br, I, at the end of which a disilylated compound of the following formula (IX) is obtained:

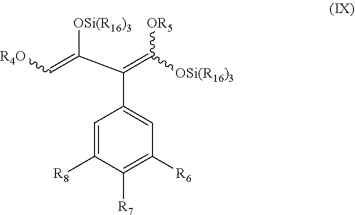

c) cyclization reaction of the compound (IX) with oxalyl chloride (ClCO)₂, at the end of which the following compound of formula (X) is obtained:

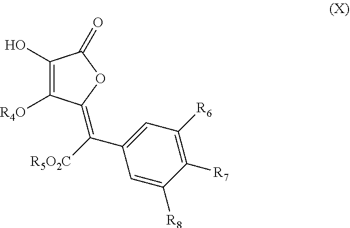

d) reaction of the compound (X) with a reagent capable of forming, by reaction with the —OH of the lactone ring, a leaving group X, at the end of which the compound of formula (V) is obtained.

By way of examples, this leaving group X may be chosen from halogens, the triflate —O—SO₂—CF₃ or the group of formula —O—SO₂—(CF₂)ₙ—CF₃ with n being an integer ranging from 1 to 8.

It should be noted that, in the above formulae, the bonds symbolized by:

[symbol]

mean that the compounds in question can exist in their different isomeric forms.

Accordingly, in step a), the reaction is carried out in a basic medium, this medium being intended to deprotonate the —CH₂— group situated at the α position with respect to the —CO₂R₅ group of the compound (VII). This basic medium may be for example a lithium diisopropylamide (or LDA) solution. The reactive species thus formed is fused with the alkyl α-alkoxyacetate to give the product (VIII). The steps b) and c), which consist in synthesizing a compound 1,3-bis(trialkylsiloxy)-1,3-butadiene (IX), followed by cyclization are adapted from the studies by Langer, such as those explicitly stated in the publication “Domino Reaction of 1,3-bis(trimethylsilyloxy)-1,3-dienes with Oxalyl Chloride: General and Stereoselective Synthesis of γ-Alkylidenebutenolides” P. Langer et al., Chem. Eur. J. 2000, 6, No. 7, 3204-3214 [6].

Finally, step d) may be considered according to any type of reaction within the capability of persons skilled in the art, the said reactions making it possible to convert the —OH group to a reactive group such as a triflate or a fluoroalkylsulphonate —SO₂—(CF₂)ₙ—CF₃, with n being an integer ranging from 1 to 8. By way of example, when the leaving group X is a triflate group (symbolized by OTf), the compound designated by the reference 5a forming part of the compounds of formula (V) may be synthesized according to the following specific reaction scheme:

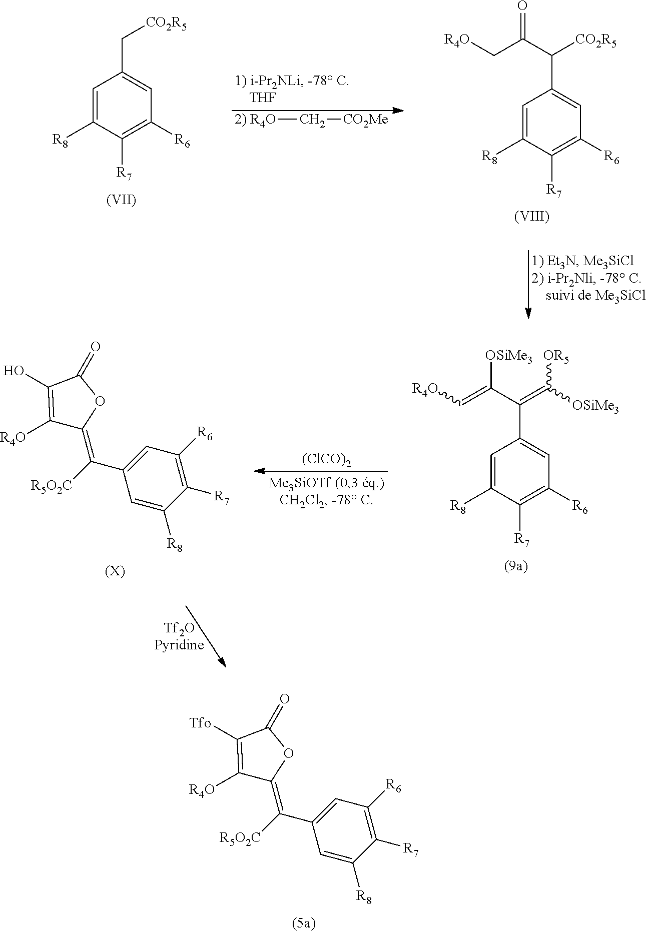

According to this specific synthesis scheme, the triflate (5a) is prepared from the corresponding alcohol (X) by treatment with triflic anhydride (Tf₂O), in the presence of a base such as pyridine. The alcohol (X) is derived from the reaction of a suitably substituted 1,3-bis(trimethylsiloxy)-1,3-butadiene (9a) with oxalyl chloride, catalyzed by methyl triflate. This cyclization reaction, and the preparation of 1,3-bis(trimethylsiloxy)-1,3-butadiene (9a) are adapted from the studies by Langer, such as those mentioned above. The β-keto ester (VIII) precursor of 1,3-bis(trimethylsiloxy)-1,3-butadiene (9a) is obtained by reaction of the lithium enolate formed from the corresponding phenyl acetate (VII) with a methyl α-alkoxyacetate.

The inventors have discovered, surprisingly, that the compounds (I) as defined in the first object, and also the excluded compounds, may be used as effective antioxidant agents for the manufacture of antioxidant compositions.

Thus, a subject of the present invention is also antioxidant agents of the following formula (I):

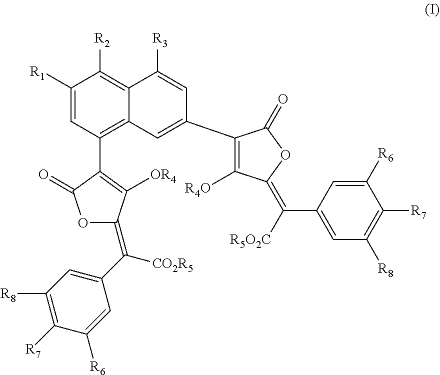

in which:
R₁, R₆, R₇ and R₈, which are identical or different, represent H, —OH or —OR₉;
R₂ represents H, —OH or —OR₉; R₃ represents H, R₉, —CO₂R₉ or —CO—NHR₁₀; or R₂ and R₃ form together —O—CO—;
R₄ and R₆, which are identical or different, represent H or R₉;
R₉ represents a linear or branched alkyl group containing from 1 to 20 carbon atoms;
R₁₀ represents R₉ or a group —(CH₂)ₐ—NH—(CH₂)_{b}—NH₂, with a and b, which are identical or different, being integers ranging from 2 to 4;
   and the salts thereof.

When R₂ and R₃ form together —O—CO—, the antioxidant agent has a chemical structure identical to that of the compound of formula (II) defined above.

Among these agents of formula (II), there may be mentioned norbadione in diacid form, for which R₁ and R₇ represent —OH, R₂ and R₃ form together —O—CO—, R₄, R₅, R₆ and R₈ represent H. Norbadione in diacid form may be represented by the following formula (XI):

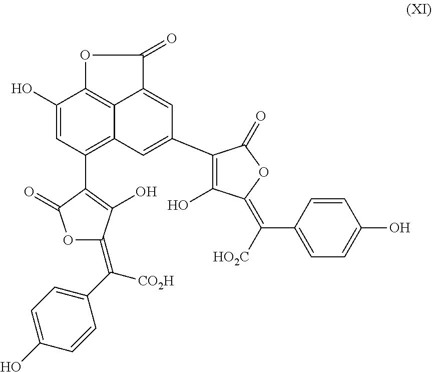

There may also be mentioned, among the antioxidant agents according to the invention, norbadione in dipotassium salt form, which may thus be represented by the following formula (XII) (which may have two forms, (a) and (b)):

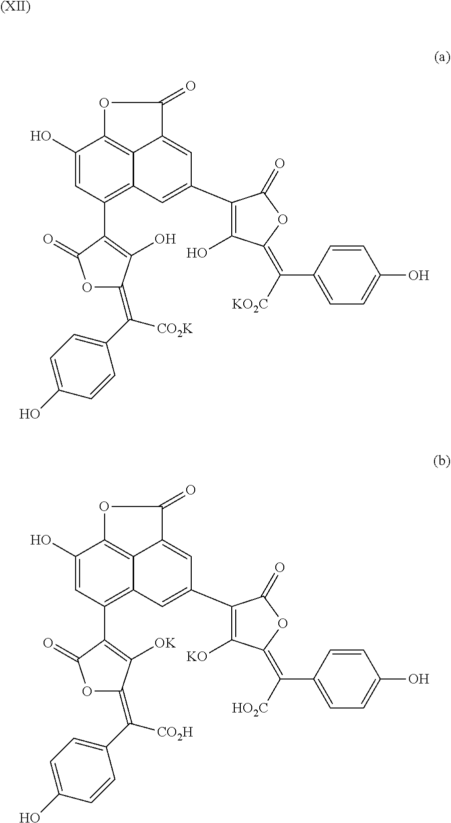

Norbadione is a natural product which has up until now been extracted from two species of fungi. It constitutes one of the cap pigments of bay boletus (Xerocomus badius), which is a very popular edible species, as is described in the article “Pigments from the cap cuticle of the Bay Boletus”, Angew. Chem. Int. Ed. Engl. 23 (1984), No. 6 [4]. It is also found in dyemaker's puffball (Pisolithus tinctorius), as indicated by the article entitled “A naphtalenoid pulvinic acid derivative from the Fungus Pisolithus Tinctorius”, Phytochemistry, vol. 24, No. 6, pp 1351-1354, 1985 [5].

Norbadione is extracted from these fungi in dipotassium salt form (formula XII) and may be converted to the corresponding diacid (formula XI) by treatment with a hydrochloric acid solution.

The inventors have surprisingly demonstrated the antioxidant activity of norbadione.

By virtue of their particularly effective antioxidant properties, the antioxidant agents of formula (I) can be used in the manufacture of antioxidant compositions, in particular of pharmaceutical compositions, of cosmetic compositions or of food compositions.

Accordingly, the invention relates to pharmaceutical compositions comprising at least one antioxidant agent according to the invention as defined above and a pharmaceutically acceptable vehicle.

The expression pharmaceutically acceptable vehicle is understood to mean a vehicle which may be administered to an individual at the same time as the antioxidant agent and which has no undesirable biological effect.

These pharmaceutical compositions may be used for the treatment of diseases resulting from oxidant stress.

Accordingly, these pharmaceutical compositions comprising the agents according to the invention may be used for the treatment of inflammatory diseases, in particular diseases resulting, in reaction to an allergen, in the production of cytokines.

For example, pharmaceutical compositions comprising norbadione in diacid form (formula XI) or disalt form (formula XII) contribute, in a dose-dependent manner, towards reducing the production of the cytokines TNF-α and IL-10 in a host infected with an allergen such as liposaccharide, which shows that the antioxidant agents according to the present invention exhibit an anti-inflammatory activity.

These pharmaceutical compositions may also be used to bring about an effect protecting cells against apoptosis following an accumulation of free radicals in the said cells.

Accordingly, these compositions may be used for treating, preventively or curatively, living cells or organisms exposed to ionizing radiation inducing the production of free radicals.

As a result, these compositions are capable of countering the effects of ionizing radiation (for example that emanating from a zone contaminated by radioactive substances) or of ultraviolet radiation on cells subjected to the said radiation, because the antioxidant agents according to the present invention incorporated into these compositions capture the radicals formed by the action of the said radiation on these living cells or organisms.

These compositions may also be used to inhibit the side effects of a medicament inducing the production of free radicals. As a result, these compositions can attenuate the cytotoxicity of medicaments, which cause, by their side effects, oxidant stress, as is the case for cisplatin.

In other words, the invention relates to the use of an antioxidant agent as defined above for the manufacture of a pharmaceutical composition intended for the treatment of inflammatory diseases.

The invention relates to the use of an antioxidant agent as defined above for the manufacture of a pharmaceutical composition intended for the treatment of a living organism exposed to ionizing radiation.

The invention finally relates to the use of an antioxidant agent as defined above for the manufacture of a medicament intended for inhibiting the side effects of a medicament inducing the production of free radicals.

It is specified that, according to the invention, the expression treatment is understood to mean a treatment which may be preventive but also curative.

Further details relating to the activity of these compositions incorporating antioxidant agents according to the invention as mentioned above will be explicitly stated in the detailed part of the description.

The subject of the present invention is also cosmetic compositions incorporating at least one antioxidant agent according to the invention.

These cosmetic compositions may be provided in various forms, such as creams, oils, intended for topical skin use, the role of the antioxidant agents being to trap the free radicals at the skin surface to which the cosmetic composition is applied. The compositions according to the invention thus contribute towards slowing the process of skin ageing caused in particular by the accumulation of free radicals.

Finally, the subject of the present invention is food compositions comprising at least one antioxidant agent according to the invention. The antioxidant agents according to the present invention may be used in particular as additives in food compositions which can produce free radicals as they age, such as oils and butter.

Particularly effective antioxidant agents which may enter into the pharmaceutical, cosmetic or food compositions correspond to the agents of formula (XI) or (XII), as defined above.

Other advantages and characteristics of the present invention will further emerge on reading the description which follows, given by way of illustration and without limitation with reference to the accompanying drawings.

### BRIEF DESCRIPTION OF THE DRAWINGS

FIGS. 1, 2 and 3 are graphs illustrating the antioxidant activity of norbadione in dipotassium salt form compared with conventional antioxidants, the said antioxidant activity being demonstrated by monitoring the degradation of thymidine subjected to oxidant stress of a radioactive nature (γ rays emitted by ¹³⁷Cs in FIG. 1 or UV rays in FIG. 2) or of a chemical nature (FIG. 3);

FIGS. 4 and 5 are graphs illustrating the anti-inflammatory effect of norbadione in diacid form (FIG. 4) or in dipotassium salt form (FIG. 5) by measuring the production of certain cytokines by cells exposed to an allergen;

FIG. 6 is a graph illustrating the protective activity of norbadione in diacid or dipotassium salt form towards cells exposed to ionizing radiation;

FIG. 7 is a graph illustrating the protective activity of norbadione in diacid or dipotassium salt form towards cells exposed to the action of cisplatin;

FIG. 8 is a graph which represents the vascular contraction of rat aorta rings C(g) (g meaning gramme) as a function of the concentration of pyrrogallol [Pyr] (μM), in the absence of norbadione and in the presence of 100 μM norbadione;

FIG. 9 is a graph representing the level of vascular relaxation of rat aorta rings R(%) as a function of the concentration of Sin−1 [Sin−1] (μM) in the absence of norbadione and in the presence of 100 μM norbadione.

### DETAILED DISCLOSURE OF THE INVENTION

Examples 1 to 5 illustrate the antioxidant properties of norbadione in diacid or disalt form.

Example 6 illustrates an example of total synthesis of a compound of formula (III) also exhibiting an antioxidant activity.

### Example 1

### Evaluation of the Antioxidant Activity of Norbadione

The evaluation of the antioxidant activity of the dipotassium salt of norbadione was carried out by a screening test in vitro.

This test is based on the monitoring of the degradation of thymidine subjected to various oxidant stresses in the presence of various antioxidant agents, which are norbadione in dipotassium salt form (called Nor-B in FIGS. 1 to 3) in accordance with the invention, and the following antioxidant agents: quercitin (1), fisetin (2), myricetin (3), catechin (4), 7-hydroxy-4-methyl-8-nitrocoumarin (5), Trolox (6) in FIGS. 1 to 3.

In the presence of an antioxidant, thymidine is protected more or less effectively according to the nature of the said antioxidant. The efficacy of the antioxidant is quantified by assaying the remaining thymidine (quantified as % on the y-axis of the graph) with the aid of an immunoenzymatic assay of the so-called “competition” ELISA type. This ELISA assay consists in producing competition, for binding to a thymidine-specific monoclonal antibody attached to a solid phase, between the thymidine remaining at the end of test and the thymidine labelled with an enzyme (acetylcholine esterase). After washing, the bound enzyme is quantified by colorimetric detection with the aid of 5,5′-dithiobis(2-nitrobenzoic acid) (termed Ellmann's reagent) and in the presence of acetylcholine.

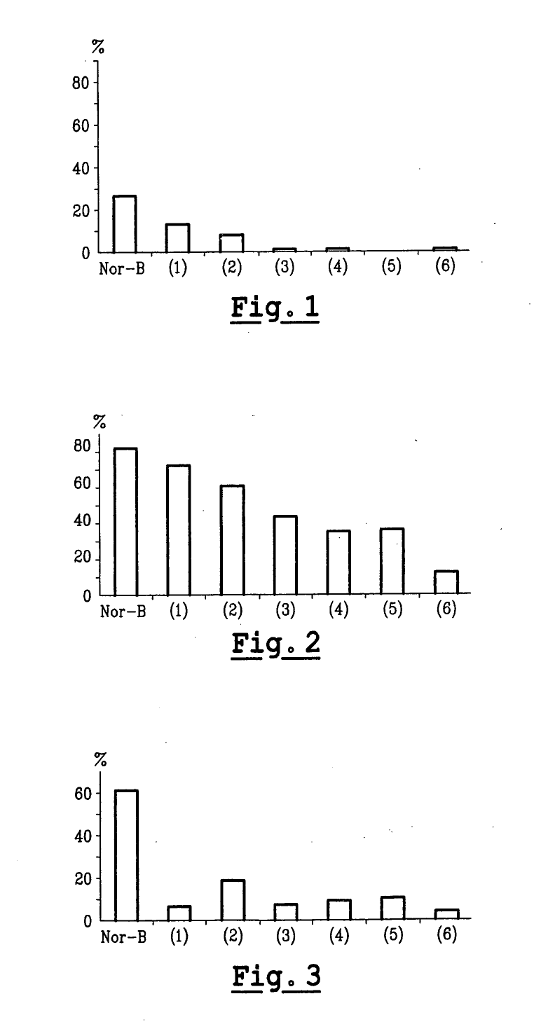

Three series of tests were used:
a first series consisting in assaying the remaining thymidine following gamma irradiation (for 3 h 30 min) induced by caesium 137, the said thymidine being present at the beginning of the test at a concentration of 15 μM with an antioxidant concentration of 12 μM (FIG. 1);
a second series consisting in assaying the remaining thymidine following ultraviolet irradiation at 254 nm (1.75 J/cm²) in the presence of 5 mM H₂O₂, the said thymidine being present at the beginning of the test at a concentration of 70 μM with an antioxidant concentration of 100 μM (FIG. 2);
a third series consisting in assaying the remaining thymidine subjected to a chemical oxidant stress (Fenton's reagent 0.35 mM FeSO₄/EDTA in the presence of 35 mM H₂O₂), the said thymidine being present at the beginning of the test at a concentration of 70 μM with an antioxidant concentration of 20 μM (FIG. 3).

The results of these tests are grouped together, for the first series in FIG. 1, for the second series in FIG. 2 and for the third series in FIG. 3, respectively.

FIGS. 1 to 3 show explicitly that norbadione has the best antioxidant power (the latter being quantified by measuring the percentage of thymidine remaining after the action of the oxidant stress, % represented on the y-axis of the graphs of FIGS. 1 to 3) compared for example with quercetin (1), obtained from tea or wine, which is reputed for its potent antioxidant property, and compared with the other conventional antioxidant agents (2), (3), (4), (5) and (6).

### Example 2

### Evaluation of the Anti-Inflammatory Power of Norbadione

In this example the anti-inflammatory biological effect induced by the antioxidant activity of norbadione in diacid or dipotassium salt form is demonstrated by detecting the cytokines produced by monocyte-type cells during treatment with an allergenic liposaccharide. This liposaccharide is an endotoxin located on the outer membrane of Gram-negative bacteria. In particular, it stimulates the production of proinflammatory cytokines by the mononuclear cells (monocytes, microphages) of the infected host.

If in the presence of the test product a change in the production of cytokines is demonstrated, it is deduced therefrom that the product has an anti-inflammatory activity.

This test is carried out in the following manner. Human blood cells from healthy donors (termed PBMC for ‘peripheral blood mononuclear cells’) are incubated with norbadione in the diacid or dipotassium salt form at increasing concentrations (from 10⁻⁸ M to 10⁻⁵ M) with or without simultaneous activation with Salmonella abortus equi lipopolysaccharide at the concentration of 5 μg·ml⁻¹, in 24-well culture plates, for 24 hours at 37° C., in a humidified atmosphere of 5% CO₂ and 95% air. After incubation, the supernatant is removed and stored at −20° C. until the test is carried out.

More specifically, the detection of the cytokines present in the samples is measured with the aid of a “sandwich” type cytometric ELISA assay. Microparticles (beads) of polystyrene (of 6 different types, each being labelled with a different quantity of fluorescent dye whose FL-3 emission wavelength is about 650 nm) are coupled to an antibody specific for one of the 2 cytokines TNFα, IL-2, IL-10. Ab-PS type antibodies are thus obtained. During incubation, the cytokines present in the sample bind to the Ab-PS antibodies. The cytokines captured are detected with the aid of a directly performed immunological test which uses 2 antibodies specific for each cytokine, coupled to phycoerythrin which emits at an FL-2 wavelength of about 585 nm (Ab-PE). After washing the excess Ab-PE, the presence of the cytokines is measured by flow cytometry.

The fluorescence is measured based on the 6 FL-3 fluorescence intensities and based on the FL-2 wavelength. The intensity of the FL-3 fluorescence makes it possible to determine the quantity of each cytokine present in the sample (in comparison with standard curves).

Further information relating to this technique is available in the article by Cook et al, Journal of Immunological Methods, 2001, 254, pages 109-118 [7].

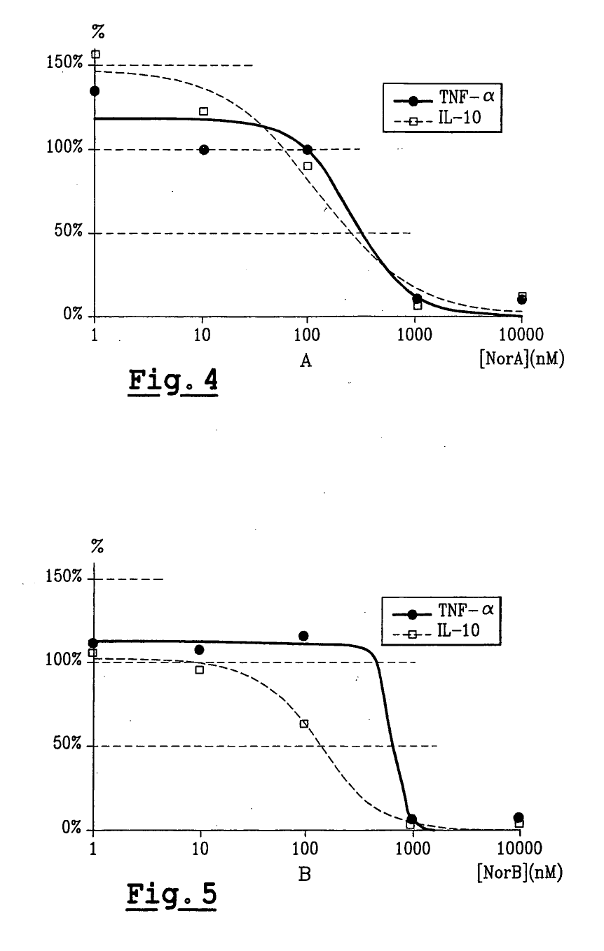

The results of these tests are grouped together in FIGS. 4 to 5, which represent the percentage response noted % (that is to say the quantity of cytokine observed relative to the quantity observed during a control where the product was not added) as a function of the concentration (in nM) of norbadione in the diacid form or dipotassium salt form, noted [Nor-A] or [Nor-B] in the said figures.

In the presence of norbadione in the diacid form (Nor-A) (FIG. 4), or of norbadione in the dipotassium salt form (Nor-B) (FIG. 5), the tests demonstrated a significant dose-dependent reduction in the production of the cytokines TNF-α and IL-10, which proves the anti-inflammatory effect of norbadione.

### Example 3

### Evaluation of the Activity of Norbadione in Protecting Cells Subjected to Ionizing Radiation

This test consists in measuring, in the presence or in the absence of norbadione in diacid or dipotassium salt form, the rate of survival of cells treated with ionizing radiation, with the aid of selective indicators for certain cellular organelles such as mitochondria.

Samples of norbadione in diacid or dipotassium salt form are added to RDM4 cell cultures (AKR mouse lymphomas) in increasing concentrations (from 0.12 to 20 μg/ml), two hours before irradiation. This is carried out by exposing microtest plates containing the cells to X rays of 15 MV, at 8 Gy, the culture medium being unchanged.

The number of living cells is determined on the sixth day by means of the Uptiblue test (which consists in measuring the micochondrial activity of the cells). The Uptiblue reagent (reazurin, also marketed under the ALAMAR BLUE trade mark) is a coloured oxidation-reduction indicator. The reazurin (blue and nonfluorescent) is reduced to resorufin (pink and fluorescent) by living cells.

Experimentally, the Uptiblue (diluted ¼ in the culture medium) is added to the cells in an amount of 20 μl per 200 μl well. After incubating for 4 hours at 37° C., the fluorescence is measured at a wavelength of 590 nm, after excitation at a wavelength of 530 nm with the aid of a fluorescence microplate reader (Fluorolite 1000, Dynex). The fluorescence intensity is proportional to the number of living cells. It is expressed as “arbitrary fluorescence unit”. This value depends on the setting of the apparatus (in particular the voltage) and the background noise (due to the fluorescence emitted by wells not containing cells, but containing Uptiblue). The latter value is subtracted from the experimental values.

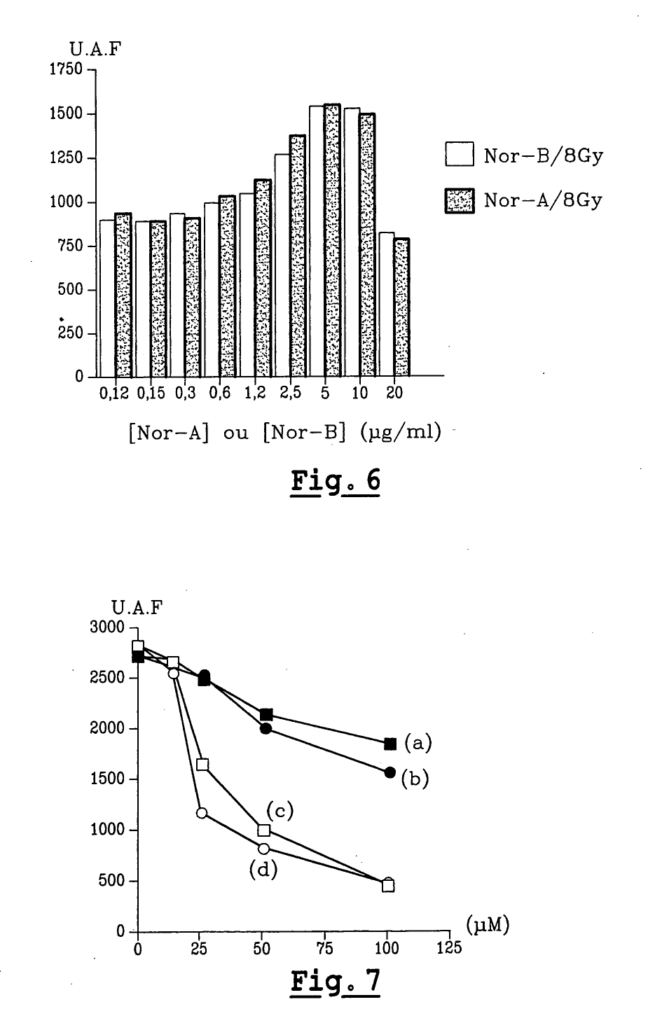

The results of this test are grouped together in FIG. 6, which represents the fluorescence observed which makes it possible to quantify the number of living cells, the said fluorescence being expressed as arbitrary fluorescence units on the y-axis of the graph (A.F.U) as a function of the concentration of norbadione in the diacid or dipotassium salt form, noted [Nor-A] or [Nor-B] (in μg/ml). In this figure, a large increase is observed in the number of living cells as a function of the norbadione concentration.

However, from a norbadione concentration of 20 μg/ml, this value decreases considerably.

It is thus deduced therefrom that norbadione, in its disalt or diacid form, protects the cells against ionizing radiation in a significant and dose-dependent manner.

It may be noted that, without irradiation, norbadione is without effect on the growth and the viability of the RDM4 cells, even at 20 μg/ml.

### Example 4

### Evaluation of the Activity of Norbadione in Protecting Cells Subjected to the Action of Cisplatin

This test consists in measuring, in the presence of norbadione in the diacid or dipotassium salt form, the rate of survival of cells treated with cisplatin.

Human thyroid carcinoma K₁ cells are cultured in flat-bottomed 96-well microtest plates in the presence of a single concentration (20 μg/ml) of norbadione in the diacid or dipotassium salt form. After two hours, a genotoxic agent, cisplatin, is added to the culture medium in increasing concentrations (from 12 to 100 μm). Two days later, the number of cells is determined by means of the sulphorhodamine B (termed SRB) test, which measures the quantity of cellular proteins.

Sulphorhodamine B (termed SRB) is an anionic dye which binds electrostatically to cellular proteins. This test is frequently used for the evaluation of the cytotoxic and cytostatic activities of novel antitumour drugs.

Further information relating to this technique is available in the article by Papazisis et al. “Optimization of the sulforhodamine B colorimetric assay”, J. Immunol. Meth, 208, pages 151-158, 1997 [8].

The results of this test are grouped together in FIG. 7, which represents the fluorescence observed after addition of SRB (expressed as arbitrary fluorescence units) as a function of the cisplatin concentration (in μM).

In this figure, it can be observed that the cytotoxicity of cisplatin is very markedly attenuated by the presence of norbadione in the disalt (curve a) or diacid (curve b) form, compared with the case where the cytotoxicity of cisplatin is evaluated in the absence of norbadione (curves c and d).

This activity for protecting cells against cisplatin results from the antioxidant activity of norbadione.

### Example 5

### Effects of Norbadione on Rat Aorta Rings

The objective of this example is to show the antioxidant activity of norbadione in the dipotassium salt form on rat aorta rings.

These series of tests demonstrate the antioxidant activity of norbadione on rat aorta rings.

During a first series of tests, rat aorta rings are subjected, in a first instance, to pyrogallol, which generates superoxide radicals, these radicals inducing contraction of the rat aorta rings and the rate of contraction of these rings is measured in the absence of norbadione.

In a second instance, the same operations are repeated, under the same conditions as those mentioned above, this time in the presence of norbadione.

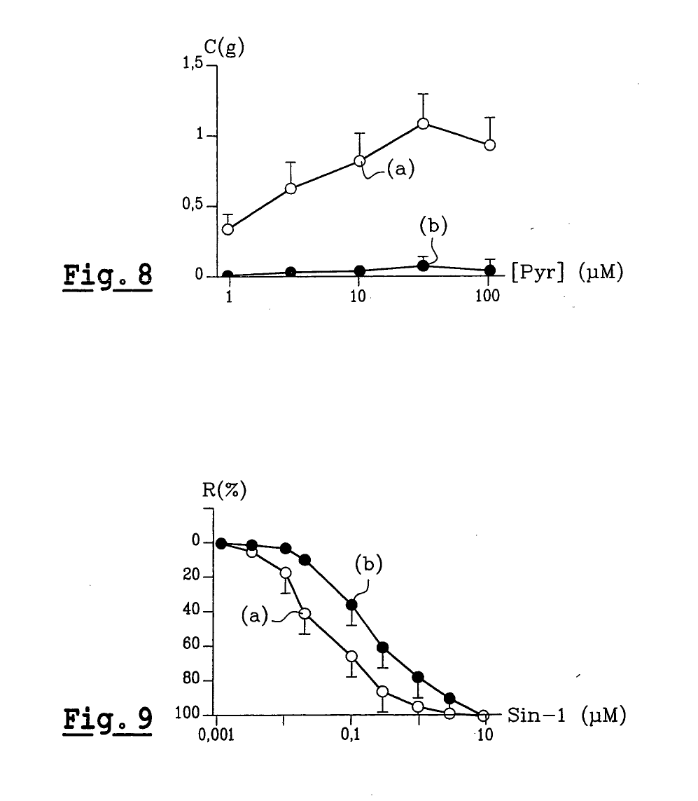

The results are grouped together in FIG. 8, which represents the rate of vascular contraction of the rings C(g) as a function of the concentration of pyrrogallol [Pyr] (μM), in the absence of norbadione (curve a) and in the presence of norbadione at the concentration of 100 μM (curve b).

It is observed that curve (a) is situated above curve (b), which means that the contraction of the aorta rings caused by pyrogallol is suppressed in the presence of norbadione because of the fact that norbadione captures the free-radical reactive oxygenated species generated by pyrogallol.

During a second series of tests, rat aorta rings are subjected to SYN-1 (3-morpholinosydnonimine), which generates NO radicals, inducing relaxation of the rat aorta rings, and the rate of relaxation of these rings is measured. In the presence of norbadione, the rate of relaxation of the rings is measured under the same conditions as those mentioned above. The results are presented in FIG. 9, which represents the rate of vascular relaxation of the rings R(%) as a function of the Sin−1 concentration [Sin−1] (μM), in the absence of norbadione (curve a) and in the presence of norbadione (100 μM) (curve b).

It is observed that curve (a) is situated below curve (b), which means that the relaxation of the aorta rings caused by SYN-1 is reduced in the presence of norbadione because of the fact that norbadione partially captures the NO radicals.

### Example 6

### Preparation of the Compound of Formula (III)

This example presents an example of preparation of a compound of formula (III):

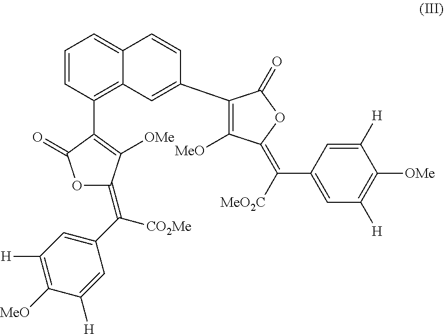

This compound is in conformity with the general definition of the compounds of formula (I), with R₁, R₂, R₃, R₆ and R₈ representing H, R₄ and R₅ representing —CH₃, R₇ representing —OCH₃.

The synthesis of this compound corresponds to the following reaction scheme:

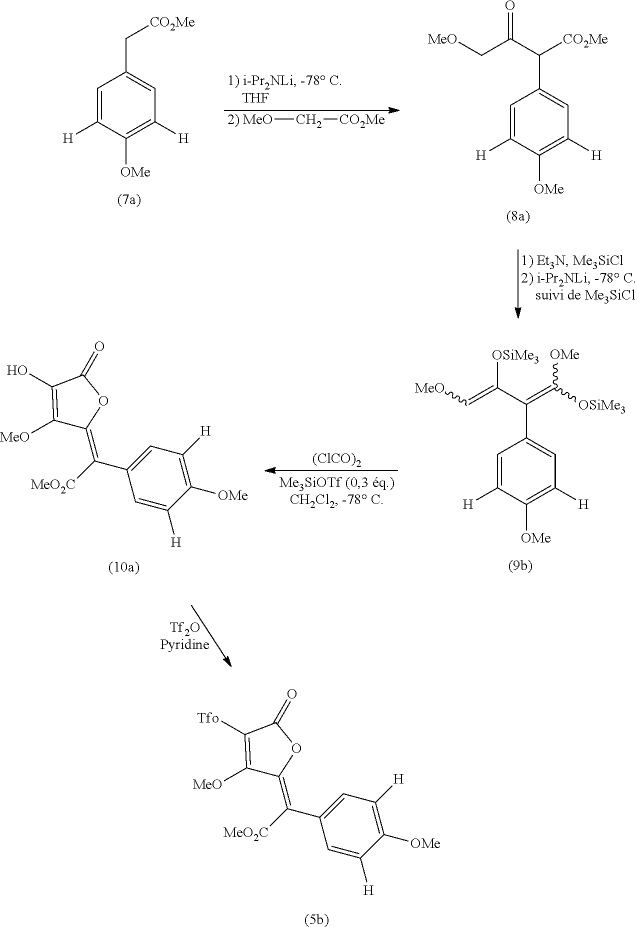

followed by a so-called “Suzuki” coupling in order to form the molecule of formula (III), the coupling being preceded by the synthesis of the compound (4a):

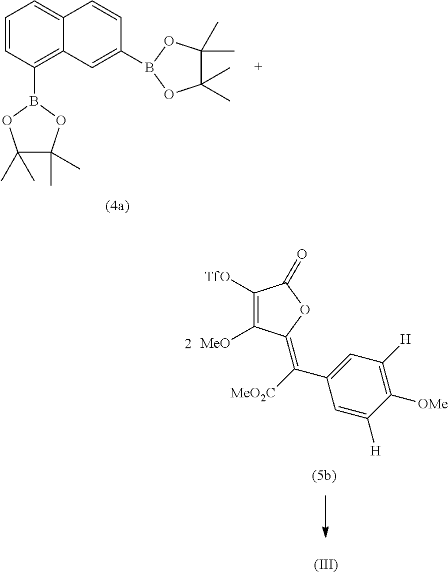

It is specified that the chemical shifts for the ¹H NMR and ¹³C NMR results are symbolized by 5 and are expressed in ppm.

### a) Preparation of the Compound (8a).

In a 100 ml two-necked flask, methyl 4-methoxyphenylacetate (7a) (4.7 ml); 29.6 mmol; 2 eq) is dissolved in THF (15 ml) and the mixture is cooled to around −70° C. A 2 M solution of lithium diisopropylamide in heptane (15 ml; 30 mmol; 2 eq) is added dropwise with a syringe and the mixture is kept for 1 hour at around −70° C. Methyl methoxyacetate (1.5 ml; 15 mmol; 1 eq) is added with a syringe and the mixture is left to react for 5 hours while returning slowly to room temperature. After hydrolysis with a saturated ammonium chloride solution, the aqueous phase is extracted 3 times with dichloromethane, dried over MgSO₄ and filtered. After evaporation, an orange-coloured oil is obtained which is chromatographed on a silica column (eluent: pentane/ethyl acetate: 8/2). 2.93 g of product are thus isolated. Appearance: yellow solid, m.p.: 44° C., yield: 77%.

¹H NMR (CDCl₃): δ=3.37 (s, 3H, OMe); 3.75 (s, 3H, OMe); 3.81 (s, 3H, CO₂Me); 4.06 and 4.10 (AB, J_{AB}=17.1 Hz, 2H, CH₂); 4.90 (s, 1H, CH); 6.91 (d, J=8.5 Hz, 2H, Ph); 7.26 (d, J=8.5 Hz, 2H, Ph).

¹³C NMR (CDCl₃): δ=52.5; 55.2; 59.3; 59.8; 69.6; 113.6; 114.2; 123.7; 130.6; 132.0; 159.5; 169.0; 202.0.

IR (KBr, cm⁻¹): 1612; 1741; 2836; 2960; 3013; 3434.

Elemental analysis (%): calculated for C₁₃H₁₆O₅: C=61.90; H=6.39. found C=62.03; H=6.44.

### b) Preparation of the Compound (9b).

The product of condensation (8a) prepared above (5.65 g; 22.4 mmol; 1 eq) is dissolved in THF (47 ml) in a 100 ml round-bottomed flask. Triethylamine (3.7 ml; 26.6 mmol; 1.2 eq) and then trimethylsilyl chloride (3.7 ml; 29 mmol; 1.3 eq) are added with a syringe. The formation of a white precipitate is immediately observed. The mixture is left to react overnight at room temperature. After evaporation of the THF, the residue is taken up in pentane. The precipitate formed is filtered on sintered glass and then on Millipore 5 μm and rinsed with pentane. After evaporation, 7.28 g of an orange-coloured oil are obtained.

The monosilylated derivative obtained (7.27 g; 22.4 mmol; 1 eq) is placed in a 100 ml round-bottomed flask and dissolved in THF (33 ml). The mixture is cooled to around −70° C. A 2 M solution of LDA (lithium diisopropylamide) in heptane (11.2 ml; 22.4 mmol; 1 eq) is added dropwise with a syringe and the mixture is kept for 1 hour at around −70° C. Trimethylsilyl chloride (3.4 ml; 26.7 mmol; 1.2 eq) is added with a syringe and the mixture is brought to room temperature over 3 hours. After evaporation of the THF, the residue is taken up in pentane and the white precipitate formed is filtered on sintered glass and then on Millipore 5 μm. After concentration, 8.52 g of an orange-coloured oil are obtained. Yield=95%. Two geometric isomers are present in this sample.

¹H NMR (CDCl₃): δ=0.04 (s, 9H, OSiMe₃ major isomer); 0.05 (s, 9H, OSiMe₃ minor isomer); 0.28 (s, 9H, OSiMe₃ minor isomer); 0.30 (s, 9H, OSiMe₃ major isomer); 3.46 (s, 3H, OMe major isomer); 3.49 (s, 3H, OMe minor isomer); 3.51 (s, 3H, OMe minor isomer); 3.56 (s, 3H, OMe major isomer); 3.80 (2s, 2*3H, OMe minor isomer and OMe major isomer); 5.62 (s, 1H, CH major isomer); 5.95 (s, 1H, CH minor isomer); 6.83 (d, J=8.5 Hz, 2H, Ph); 7.24-7.31 (m, 2H, Ph).

### c) Preparation of the Compound (10a).

In a 1 l three-necked flask, the bis-silylated derivative (9b) prepared above (8.52 g; 21.4 mmol; 1 eq) is dissolved in dichloromethane (400 ml) and the mixture is cooled to around −70° C. Oxalyl chloride (2.5 ml; 28.6 mmol; 1.3 eq) is added with a syringe and the medium becomes orange. A solution of trimethylsilyl triflate (1.2 ml; 6.6 mmol; 0.3 eq) in dichloromethane (120 ml) is poured in with a dropping funnel over one hour. The cold bath is saturated with dry ice for the reaction medium to return very slowly to room temperature overnight. After hydrolysis with a saturated NaCl solution, the mixture is extracted 3 times with dichloromethane, dried over MgSO₄, filtered and concentrated. 7.95 g of a brown solid are recovered. After chromatography on a silica column (eluent: ethyl acetate/pentane: 1/1), 3.53 g of product (5′) are obtained. Appearance: yellow solid, m.p.=160° C., yield: 54%, isomer (E).

¹H NMR (CDCl₃): δ=3.85 (s, 3H, OMe or CO₂Me); 3.87 (s, 3H, CO₂Me or OMe); 4.16 (s, 3H, OMe phenyl); 6.96-9.99 (m, 2H, Ph); 7.47-7.50 (m, 2H, Ph).

¹³C NMR (acetone-d₆): δ=52.3; 55.2; 59.8; 113.5; 114.5; 124.6; 130.6; 131.7; 139.9; 143.3; 160.4; 164.5; 167.3.

IR (KBr, cm⁻¹): 1667; 1731; 3239; 3295.

Elemental analysis (%): calculated for C₁₅H₁₄O₇: C=58.82; H=4.61. found C=59.07; H=4.92.

### d) Preparation of the Compound (5b).

In a 250 ml round-bottomed flask, the alcohol (10a) prepared above (3.24 g; 10.6 mmol; 1 eq) is suspended in dichloromethane (100 ml) and the mixture is cooled to around −70° C. Pyridine (2.2 ml; 27 mmol; 2.5 eq) and then triflic anhydride (2.2 ml; 13 mmol; 1.2 eq) are added with a syringe. After returning to room temperature over 6 hours, the mixture is hydrolyzed, washed 3 times with water, dried over MgSO₄, filtered and concentrated. After chromatography on a silica column (eluent: dichloromethane), 3.14 g of triflate (VI) are isolated. Appearance: yellow solid; m.p.=93° C.; yield: 68%, isomer (E).

¹H NMR (CDCl₃): δ=3.85 (s, 3H, OMe); 3.90 (s, 3H, OMe); 4.29 (s, 3H, OMe); 6.94 (d, J=9.2 Hz, 2H, Ph).

¹³C NMR (CDCl₃): δ=53.1; 55.5; 61.1; 114.6; 116.4; 120.6; 122.6; 129.4; 131.4; 135.7; 155.8; 161.3; 166.1.

Mass [m/z (%)]: 456 (100) (M+NH₄*); 439 (11) (M+1).

IR (KBr, cm⁻¹): 1604; 1643; 1667; 1733; 1744; 1783; 1802; 2961.

### e) Preparation of the Compound (4a).

The bistriflate derived from 1,7-dihydroxynaphthalene (157 mg; 0.98 mmol), pinacolborane (0.44 ml; 3 mmol), PdCl₂ (44 mg; 0.06 mmol), triethylamine (0.84 ml; 6 mmol) and dioxane degassed beforehand (8 ml) are introduced into a 50 ml two-necked flask and placed under an argon atmosphere. The mixture is heated under reflux for 2 hours, with stirring. After hydrolysis with 20 ml of water, the aqueous phase is extracted 3 times with dichloromethane. The organic phases are combined, dried over MgSO₄, filtered and concentrated. Chromatography on a silica column (eluent: hexane/ethyl acetate: 8/2) makes it possible to isolate 104 mg of boron-containing derivative; yield=43%.

¹H NMR (CDCl₃): δ=1.43 and 1.49 (2s, each 12H, Me); 7.55 (t, 1H, J=6.7 Hz, g); 7.88 (m, 2H); 7.97 (d, 1H, J=6.7 Hz); 8.09 (d, 1H, J=6.7 Hz); 9.31 (s, 1H).

¹³C NMR (CDCl₃): δ=24.8; 83.6; 126.9; 128.2; 125.7; 127.2; 129.8; 130.1; 130.9; 134.7; 136.0; 136.5.

### f) Preparation of the Final Compound (III).

The triflate (5b) prepared above (214 mg; 0.488 mmol; 2.1 eq), 1,7-bis(4,4,5,5-tetramethyl-1,3,2-dioxaborolan-2-yl)naphthalene (89 mg; 0.234 mmol; 1 eq) (4b), dichlorobis(triphenylphosphine)palladium (19 mg; 0.003 mmol; 0.1 eq) and 20 ml of THF degassed beforehand are introduced into a 50 ml two-necked flask and placed under an argon atmosphere. A 2 M aqueous sodium bicarbonate solution (4.8 ml) is added. The mixture is heated under reflux for 3 hours. After hydrolysis with 30 ml of water, the aqueous phase is extracted 3 times with dichloromethane. The organic phases are combined, dried over MgSO₄, filtered and concentrated. Chromatography on a silica column (eluent: hexane/ethyl acetate: 6/4) makes it possible to isolate 104 mg of compound ( ). Appearance: yellow solid. Yield=63%.

¹H NMR (CDCl₃): δ=3.63 (s, 3H, OMe enolic); 3.80 (s, 3H, OMe phenolic; 3.86; 3.87; 3.90; 3.92 (4s, 3H each, OMe phenolic, CO₂Me); 6.94-6.99 (m, 4H, CHCOMe); 7.57-7.59 (m, 2H); 7.66-7.74 (m, 5H); 7.90 (s, 1H); 7.96-7.99 (m, 2H).

¹³C NMR (CDCl₃): δ=52.6; 55.2; 60.6; 61.5; 103.4; 106.5; 114.1; 116.9; 123.2; 125.9; 126.5; 127.3; 127.5; 128.7; 129.3; 130.1; 130.6; 130.7; 131.3; 133.1; 139.4; 139.5; 160.3; 163.0; 164.0; 166.8; 168.0.

Elemental analysis (%): calculated for C₄₀H₃₂O₁₂: C=68.18; H=4.58. found C=68.03; H=4.83.

### REFERENCES CITED

[1] M. Jang et al., “Cancer Chemoprotective Activity of Resveratrol, a natural product derived from grapes”, Science 1997, 275, 218-220;
[2] M. V. Eberhardt et al., “Antioxydant Activity of Fresh Apples”, Nature 2000, 405, 903-904;
[3] T. Finkel et al., “Oxidants, Oxidative Stress and the biology of ageing”, Nature 2000, 408, 239-247;
[4] “Pigments from the cap cuticle of the Bay Boletus”, Angew. Chem. Int. Ed. Engl. 23 (1984), No. 6;
[5] “A naphtalenoid pulvinic acid derivative from the Fungus Pisolithus Tinctorius”, Phytochemistry, vol. 24, No. 6, pp 1351-1354, 1985;
[6] P. Langer et al., “Domino Reaction of 1,3-bis(trimethylsilyloxy)-1,3-dienes with Oxalyl Chloride: General and Stereoselective Synthesis of γ-Alkylidenebutenolides” Chem. Eur. J. 2000, 6, No. 7, 3204-3214;
[7] Cook et al., Journal of Immunological Methods, 2001, 254, pages 109-118;
[8] Papazisis et al., “Optimization of the sulforhodamine B colorimetric assay”, J. Immunol. Meth, 208, pages 151-158, 1997 [8].

1. Compound corresponding to the following formula (I): 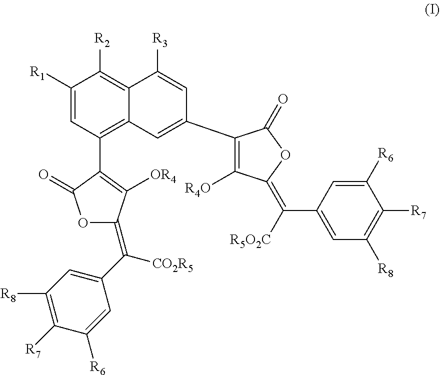
in which:
R₁, R₆, R₇ and R₈, which are identical or different, represent H, —OH or —OR₉;
R₂ represents H, —OH or —OR₉; R₃ represents H, R₉, —CO₂R₉ or —CO—NHR₁₀; or R₂ and R₃ form together —O—CO—;
R₄ and R₅, which are identical or different, represent H or R₉;
R₉ represents a linear or branched alkyl group containing from 1 to 20 carbon atoms;
R₁₀ represents R₉ or a group —(CH₂)ₐ—NH—(CH₂)_{b}—NH₂, with a and b, which are identical or different, being integers ranging from 2 to 4;
and the salts of these compounds;
with the exception:
of the compound for which R₂ and R₃ form together a group —OCO—, R₄, R₅, R₆ and R₈ represent H, R₁ and R₇ represent —OH and the disalts of potassium corresponding to this compound;
of the compound in which R₂ and R₃ form together a group —O—CO—, R₁ and R₇ represent —OCH₃, R₄ and R₅ represent —CH₃ and R₆ and R₈ represent H;
of the compound in which R₁, R₂ and R₇ represent —O—CH₃, R₃ represents —CO₂CH₃, R₄ and R₅ represent CH₃ and R₆ and R₈ represent H.

2. Compound according to claim 1, for which R₂ and R₃ form together a group —O—CO—, the said compound corresponding to the following formula (II): 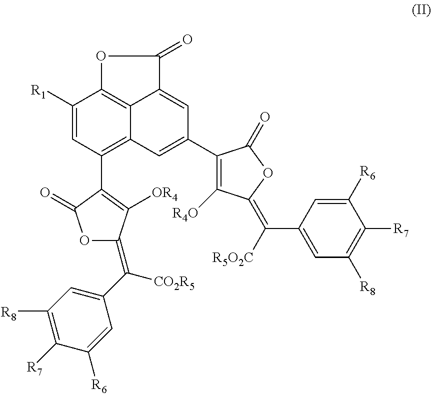
R₁, R₄, R₅, R₆, R₇ and R₈ having the same definition as that given in claim 1.

3. Compound corresponding to the following formula (III):

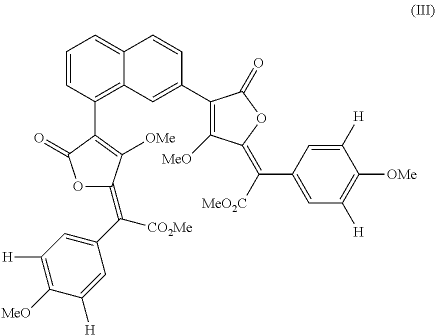

4. Method for preparing a compound of the following formula (I): 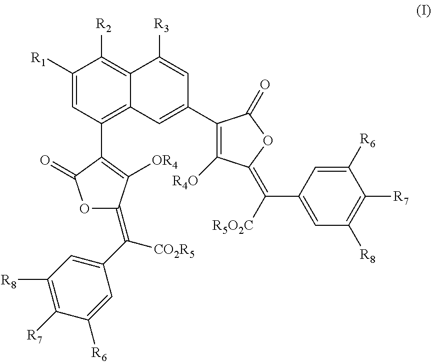
in which:
R₁, R₆, R₇ and R₈, which are identical or different, represent H, —OH or —OR₉;
R₂ represents H, —OH or —OR₉; R₃ represents H, R₉, —CO₂R₉ or —CO—NHR₁₀; or R₂ and R₃ form together —O—CO—;
R₄ and R₅, which are identical or different, represent H or R₉;
R₉ represents a linear or branched alkyl group containing from 1 to 20 carbon atoms;
R₁₀ represents R₉ or a group —(CH₂)ₐ—NH—(CH₂)_{b}—NH₂, with a and b, which are identical or different, being integers ranging from 2 to 4;
and the salts of these compounds,
the said method comprising successively:
a step consisting in reacting a compound of the following formula (IV): 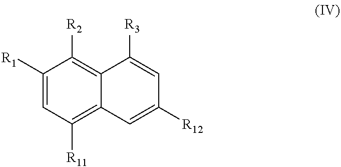
in which:
R₁, R₂ and R₃ have the same definition as that given in claim 1;
R₁₁ and R₁₂ independently represent —B(OR₁₃)(OR₁₄) or —Sn(R₁₅)₃;
R₁₃ and R₁₄, which are identical or different, represent H or an alkyl group of 1 to 7 carbon atoms or R₁₃ and R₁₄ form together a linear or branched alkylene group;
R₁₅ represents a methyl or butyl group,
with a compound of the following formula (V): 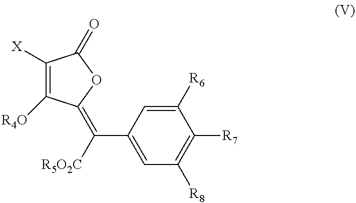
in which:
R₄, R₅, R₆, R₇ and R₈ correspond to the same definition as that given in claim 1;
X represents a leaving group,
the said reaction being carried out in the presence of a base and a catalyst based on platinum or palladium; and
optionally, a step of treatment intended to obtain a salt corresponding to the compound of formula (I).

5. Method of preparation according to claim 4, in which the platinum-based catalyst is dichlorobis(triphenylphosphine)palladium.

6. Method of preparation according to claim 4, in which the intermediate compound (IV), with R₁₁ and R₁₂ representing —B(OR₁₃)(OR₁₄), is prepared by reacting a naphthalene-derived compound of formula (VI): 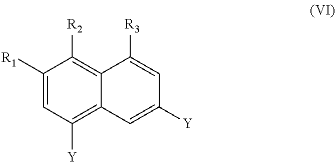
in which:
R₁, R₂ and R₃ have the same definition as that given in claim 1;
the Y's, which are identical or different, represent leaving groups;
with a boron compound corresponding to one of the following formulae: 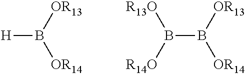
R₁₃ and R₁₄ having the same meaning as that given in claim 4,
the said reaction being carried out in the presence of a base and a platinum- or palladium-based catalyst.

7. Method of preparation according to claim 4, in which the intermediate compound (V) is prepared by the following succession of steps:
a) reaction of a phenyl acetate of the following formula (VII): 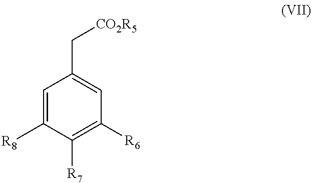
R₅, R₆, R₇ and R₈ having the same definition as that given in claim 1, in a basic medium, with an alkyl α-alkoxyacetate of formula R₄O—CH₂—CO—OAlk, R₄ corresponding to the same definition as that given in claim 1, the Alk group being a linear or branched alkyl group containing from 1 to 20 carbon atoms, at the end of which a compound of the following formula (VIII) is obtained: 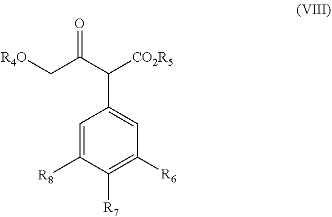
b) reaction of the compound (VIII), in a basic medium, with a silylated compound of formula (R₁₆)₃SiHal, R₁₆ being a linear or branched alkyl group containing from 1 to 4 carbon atoms, Hal being a halogen group such as F, Cl, Br, I, at the end of which a disilylated compound of the formula (IX) is obtained: 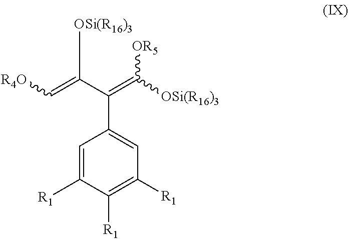
c) cyclization reaction of the compound (IX) with oxalyl chloride (ClCO)₂, at the end of which the compound of formula (X) is obtained: 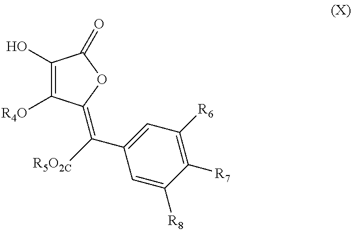
d) reaction of the compound (X) with a reagent capable of forming, by reaction with the —OH of the lactone ring, a leaving group X, at the end of which the compound of formula (V) is obtained.

8. Antioxidant agent of the following formula (I): 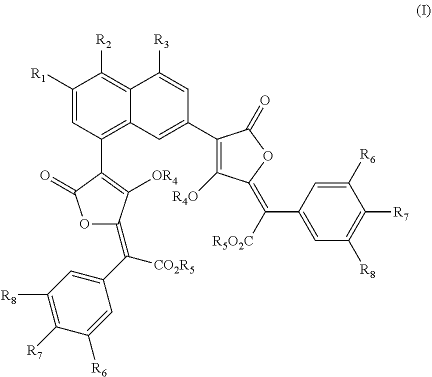
in which
R₁, R₆, R₇ and R₈, which are identical or different, represent H, —OH or —OR₉;
R₂ represents H, —OH or —OR₉;
R₃ represents H, R₉, —CO₂R₉ or —CO—NHR₁₀;
or
R₂ and R₃ form together —O—CO—;
R₄ and R₅, which are identical or different, represent H or R₉;
R₉ represents a linear or branched alkyl group containing from 1 to 20 carbon atoms;
R₁₀ represents R₉ or a group —(CH₂)ₐ—NH—(CH₂)_{b}—NH₂, with a and b, which are identical or different, being integers ranging from 2 to 4;
and the salts thereof.

9. Antioxidant agent according to claim 8, for which R₂ and R₃ form together a group —O—CO—, corresponding to the following formula (II): 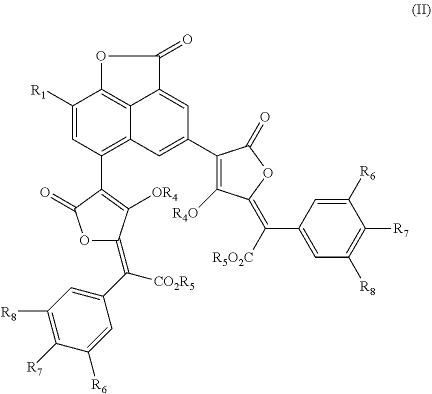
R₁, R₄, R₅, R₆, R₇ and R₈ having the same definition as that given in claim 8.

10. Antioxidant agent according to claim 8, in which R₄, R₅, R₆ and R₈ correspond to a hydrogen atom, R₁ and R₇ represent —OH, the said compound corresponding to the following formula (XI):

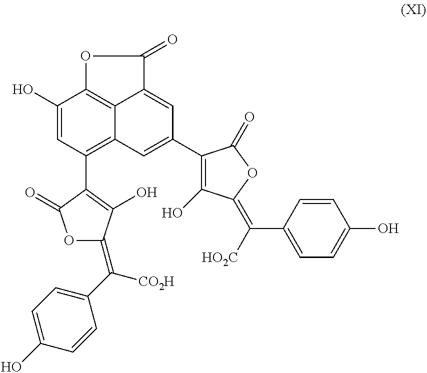

11. Antioxidant agent according to claim 8, corresponding to the dipotassium salt of the compound of formula (XI) of claim 10, the said disalt existing in two forms, (a) and (b) corresponding to the following formula (XII):

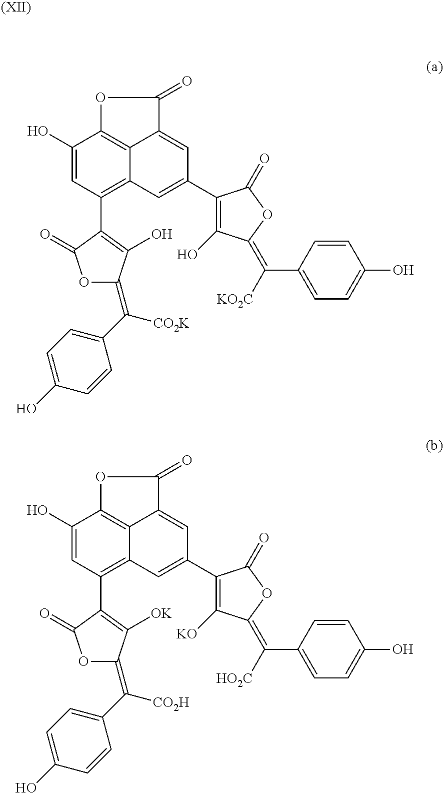

12. Pharmaceutical composition comprising at least one antioxidant agent according to claim 8 and a pharmaceutically acceptable vehicle.

13. Cosmetic composition comprising at least one antioxidant agent according to claim 8.

14. Food composition comprising at least one antioxidant agent according to claim 8.

15. Use of an antioxidant agent as defined in claim 8 for the manufacture of a pharmaceutical composition intended for the treatment of inflammatory diseases.

16. Use of an antioxidant agent as defined in claim 8 for the manufacture of a pharmaceutical composition intended for the treatment of a living organism exposed to ionizing radiation inducing the production of free radicals.

17. Use of an antioxidant agent as defined in claim 8 for the manufacture of a pharmaceutical composition intended for inhibiting the side effects of a medicament inducing the production of free radicals.
